(19) United States
(12) Patent Application Publication
(10) Pub. No.: US 2025/0334541 A1
(43) Pub. Date: Oct. 30, 2025

(54) SENSOR ELEMENT, GAS SENSOR, EVALUATION METHOD FOR SENSOR ELEMENT, AND PROGRAM

(71) Applicant: NGK INSULATORS, LTD., Nagoya-shi (JP)

(72) Inventor: Shiho IWAI, Nagoya-shi (JP)

(21) Appl. No.: 19/264,100

(22) Filed: Jul. 9, 2025

Related U.S. Application Data
(63) Continuation of application No. PCT/JP2024/000436, filed on Jan. 11, 2024.

(30) Foreign Application Priority Data
Jan. 19, 2023 (JP) 2023-006323

(51) Int. Cl. G01N 27/407; G01N 27/41; G01N 27/416; G01N 33/00

(52) U.S. Cl. CPC G01N 27/4072; G01N 27/41; G01N 27/4163; G01N 33/0027

(57) ABSTRACT

A sensor element includes an element body, at least one adjustment pump cell having an inner electrode disposed in an oxygen concentration adjustment chamber, a measurement electrode, a diffusion rate-limiting section. A height t [mm] of the diffusion rate-limiting section, obtained based on the following parameters: a path length L [cm] of the diffusion rate-limiting section; a width H [cm] of the diffusion rate-limiting section; a limiting current Ip [A] of the adjustment pump cell; the Faraday constant F [A·sec/mol]; the diffusion coefficient D [cm²/sec] of oxygen; the gas constant R [cm³·atm/mol·K]; a temperature T [K] of the inner electrode; an oxygen partial pressure Poe [atm] in the measurement gas; and an oxygen partial pressure Pod [atm] in the oxygen concentration adjustment chamber, is 0.0035 or greater.

### CROSS-REFERENCE TO RELATED APPLICATION

This application is a continuation application of PCT/JP2024/000436, filed on Jan. 11, 2024, which claims the benefit of priority of Japanese Patent Application No. JP2023-006323, filed on Jan. 19, 2023, the entire contents of which are incorporated herein by reference.

### BACKGROUND OF THE INVENTION

### 1. Field of the Invention

The present invention relates to a sensor element, a gas sensor, an evaluation method for a sensor element, and a program.

### 2. Description of the Related Art

Conventionally, a gas sensor configured to detect a concentration of a specific gas, such as NOx, in a measurement gas, such as exhaust gas from an automobile, is known. For example, PTL 1 discloses a gas sensor including a laminated body with a plurality of oxygen-ion-conductive solid electrolyte layers and a gas flow path provided therein, configured to introduce and circulate the measurement gas from a gas inlet. The gas sensor also includes a main pump cell with an inner pump electrode disposed in a first internal cavity within the gas flow path and an outer pump electrode disposed on the outer surface of the laminated body, a measurement electrode disposed downstream of the first internal cavity within the gas flow path, and a slit-shaped diffusion rate-limiting section, which is provided in the gas flow path and configured to guide the measurement gas from the outside into the first internal cavity while imparting diffusion resistance. When detecting the NOx concentration using this gas sensor, a pump current Ip0 is first applied between the inner and outer pump electrodes to adjust the oxygen concentration in the first internal cavity. Next, NOx contained in the measurement gas, after the oxygen concentration has been adjusted, is reduced in the second internal cavity. The NOx concentration in the measurement gas is then detected based on a pump current Ip2 that flows when the oxygen in the second internal cavity is pumped out.

### CITATION LIST

### Patent Literature

PTL 1: JP 2014-209128 A

### SUMMARY OF THE INVENTION

In such gas sensors, the diffusion resistance from the outside up to the first internal cavity may become excessively large due to the cross-sectional shape of the diffusion rate-limiting section. This may result in an excessive increase in the dependence (static pressure dependence) of the pump current Ip0 of the main pump cell (adjustment pump cell) on the pressure of the measurement gas. Therefore, there is a need for a gas sensor capable of suppressing an excessive increase in the static pressure dependence of the pump current Ip0.

The main object of the sensor element, gas sensor, evaluation method for the sensor element, and program according to the present invention is to provide a sensor element capable of suppressing an excessive increase in the static pressure dependence of the pump current of the adjustment pump cell.

In order to achieve the above main object, the sensor element, gas sensor, evaluation method for the sensor element, and program according to the present invention employ the following configuration.

[1] A sensor element according to the present invention is a sensor element configured to detect a concentration of a specific gas in a measurement gas, the sensor element comprising: an element body having an oxygen-ion-conductive solid electrolyte layer and a measurement gas flow path provided therein, the measurement gas flow path configured to introduce and circulate the measurement gas; at least one adjustment pump cell having an inner electrode disposed in an oxygen concentration adjustment chamber within the measurement gas flow path, the adjustment pump cell being configured to adjust an oxygen concentration in the oxygen concentration adjustment chamber; a measurement electrode disposed in a measurement chamber located downstream of the oxygen concentration adjustment chamber within the measurement gas flow path; and a diffusion rate-limiting section provided in the measurement gas flow path, the diffusion rate-limiting section being configured to guide the measurement gas from the outside into the oxygen concentration adjustment chamber while imparting diffusion resistance thereto; wherein a height t [mm] of the diffusion rate-limiting section, obtained using Equation (A) based on the following parameters: a path length L [cm] of the diffusion rate-limiting section; a width H [cm] of the diffusion rate-limiting section; a limiting current Ip [A] of the adjustment pump cell; the Faraday constant F [A·sec/mol]; the diffusion coefficient D [cm²/sec] of oxygen; the gas constant R [cm³ atm/mol-K]; a temperature T [K] of the inner electrode; an oxygen partial pressure Poe [atm] in the measurement gas; and an oxygen partial pressure Pod [atm] in the oxygen concentration adjustment chamber, is 0.0035 or greater.

$$t = L / H \times Ip \times 1 / ( 4 \times F \times D / ( R \times T ) ) \times 1 / ( Poe - Pod ) \times 10 \qquad ( A )$$

The sensor element according to the present invention is configured such that the height t [mm] of the diffusion rate-limiting section, obtained using Equation (A) based on the following parameters: the path length L [cm] of the diffusion rate-limiting section, the width H [cm] of the diffusion rate-limiting section, the limiting current Ip [A] of the adjustment pump cell, the Faraday constant F [A·sec/mol], the diffusion coefficient D [cm²/sec] of oxygen, the gas constant R [cm³·atm/mol·K], the temperature T [K] of the inner electrode, the oxygen partial pressure Poe [atm] in the measurement gas, and the oxygen partial pressure Pod [atm] in the oxygen concentration adjustment chamber, is 0.0035 or greater. This configuration allows suppression of an excessive reduction in the cross-sectional area [cm²], which is the product of the width H [cm] of the diffusion rate-limiting section and the height t/10 [cm]. In other words, it prevents an excessive increase in a diffusion resistance from the outside up to the oxygen concentration adjustment chamber. Therefore, an excessive increase in a static pressure dependence of the pump current of the adjustment pump cell can be suppressed. The inventors have confirmed this through experiments and analyses. As a result, it is possible to provide a sensor element capable of suppressing the excessive increase in the static pressure dependence of the pump current of the adjustment pump cell. In cases where the element body is manufactured by laminating and further firing multiple solid electrolyte layers to integrate them, the central portion in the width direction of the diffusion rate-limiting section may bulge or dent related to both end portions, and the height of the diffusion rate-limiting section often becomes non-uniform at different positions in the width direction. Therefore, it is difficult to directly measure the height of the diffusion rate-limiting section. In contrast, by using the above Equation (A), the height t (average height) of the diffusion rate-limiting section can be calculated.

[2] In the sensor element according to the present invention (the sensor element described in [1] above), the height t may be 0.0090 or greater. This configuration allows further suppression of the excessive increase in the static pressure dependence of the pump current of the adjustment pump cell.

[3] In the sensor element according to the present invention (the sensor element described in [1] or [2] above), the height t may be 0.0250 or less. This configuration allows suppression of the degradation rate of the adjustment pump cell. The inventors have confirmed this through experiments and analyses.

[4] In the sensor element according to the present invention (the sensor element according to any one of [1] to [3] above), the diffusion rate-limiting section may include first to n-th (n≥2) diffusion rate-limiting sections, the L/H may be obtained as a sum of Li/Hi (i: 1 to n), using path lengths Li [cm] and widths Hi [cm] of the respective first to n-th diffusion rate-limiting sections, and the height t may be an average of the heights ti of the respective first to n-th diffusion rate-limiting sections. Here, the height ti of the i-th diffusion rate-limiting section corresponds to the height of a single slit when the i-th diffusion rate-limiting section includes only one slit of a path length Li and a width Hi, and corresponds to the combined height of the slits when the i-th diffusion rate-limiting section includes a plurality of slits of the path length Li and the width Hi.

[5] In the sensor element according to the present invention (the sensor element according to any one of [1] to [4] above), a plurality of adjustment pump cells, each having the oxygen concentration adjustment chamber and the inner electrode, may be provided in series along the measurement gas flow path, the diffusion rate-limiting section may be provided upstream of the most upstream oxygen concentration adjustment chamber within the measurement gas flow path, and the limiting current Ip may be the limiting current of the adjustment pump cell that adjusts the oxygen concentration in the most upstream oxygen concentration adjustment chamber within the measurement gas flow path. In this case, the excessive increase in the static pressure dependence of the pump current of the most upstream oxygen concentration adjustment chamber within the measurement gas flow path can be suppressed.

[6] The gas sensor according to the present invention includes the sensor element described in any one of [1] to [5] above. Therefore, the gas sensor according to the present invention can achieve the same effects as the sensor element described above, such as the effect of providing the sensor element capable of suppressing the excessive static pressure dependence of the pump current of the adjustment pump cell.

[7] An evaluation method for a sensor element is an evaluation method configured to detect a concentration of a specific gas in a measurement gas, the sensor element comprising: an element body having an oxygen-ion-conductive solid electrolyte layer and a measurement gas flow path provided therein, the measurement gas flow path configured to introduce and circulate the measurement gas; at least one adjustment pump cell having an inner electrode disposed in an oxygen concentration adjustment chamber within the measurement gas flow path, the adjustment pump cell being configured to adjust an oxygen concentration in the oxygen concentration adjustment chamber; a measurement electrode disposed in a measurement chamber located downstream of the oxygen concentration adjustment chamber within the measurement gas flow path; and a diffusion rate-limiting section provided in the measurement gas flow path, the diffusion rate-limiting section being configured to guide the measurement gas from the outside into the oxygen concentration adjustment chamber while imparting diffusion resistance thereto; wherein the evaluation method for the sensor element under evaluation executes: (a) a step of calculating a height t [mm] of the diffusion rate-limiting section using Equation (B) based on a path length L [cm] of the diffusion rate-limiting section, a width H [cm] of the diffusion rate-limiting section, a limiting current Ip [A] of the adjustment pump cell, the Faraday constant F [A·sec/mol], the diffusion coefficient D [cm²/sec] of oxygen, the gas constant R [cm³·atm/mol·K], a temperature T [K] of the inner electrode, an oxygen partial pressure Poe [atm] in the measurement gas, and an oxygen partial pressure Pod [atm] in the oxygen concentration adjustment chamber; and (b) a step of evaluating using the height t.

$$t = L / H \times Ip \times 1 / ( 4 \times F \times D / ( R \times T ) ) \times 1 / ( Poe - Pod ) \times 10 \qquad ( B )$$

In the evaluation method for the sensor element according to the present invention, the evaluation method, for the sensor element under evaluation, calculates the height t [mm] of the diffusion rate-limiting section using Equation (B) based on the path length L [cm] of the diffusion rate-limiting section, the width H [cm] of the diffusion rate-limiting section, the Faraday constant F [A·sec/mol], the diffusion coefficient D [cm²/sec] of oxygen, the gas constant R [cm³ atm/mol-K], the temperature T [K] of the inner electrode, the limiting current Ip [A] of the adjustment pump cell, the oxygen partial pressure Poe [atm] in the measurement gas, and the oxygen partial pressure Pod [atm] in the oxygen concentration adjustment chamber. Subsequently, the evaluation method evaluates the sensor element under evaluation using the height t. This allows the evaluation of whether the cross-sectional area [cm²], which is the product of the width H [cm] of the diffusion rate-limiting section and the height t/10 [cm], is excessively small. In other words, it enables the evaluation of whether the diffusion resistance from the outside up to the oxygen concentration adjustment chamber is excessively large. Therefore, it is possible to evaluate whether static pressure dependence of the pump current of the adjustment pump cell is excessively high. The inventors have confirmed this through experiments and analyses. As a result, it is possible to provide a sensor element capable of suppressing the excessive increase in the static pressure dependence of the pump current of the adjustment pump cell.

[8] The program according to the present invention causes one or more computers to execute each step of the evaluation method for the sensor element according to the present invention (the evaluation method of the sensor element described in [7] above). This program may be recorded on a computer-readable recording medium (e.g. a hard disk, SSD, ROM, FD, CD, DVD, etc.), may be distributed from one computer to another via a transmission medium (such as a communication network like the Internet or LAN), or may be transferred in other forms. Execution of the program according to the present invention on one or more computers causes each step of the evaluation method for the sensor element according to the present invention to be executed. Therefore, the program according to the present invention can achieve the same effects as the evaluation method for the sensor element according to the present invention, such as the effect of providing the sensor element capable of suppressing the excessive increase in the static pressure dependence of the pump current of the adjustment pump cell.

### BRIEF DESCRIPTION OF THE DRAWINGS

FIG. 1 is a schematic cross-sectional view schematically showing an example of a configuration of a gas sensor 100.

FIG. 2 is an enlarged view showing an area around the first and second diffusion rate-limiting sections 11 and 13 of the gas sensor 100.

FIG. 3 is a block diagram showing the electrical connection relationship between a control device 95 and respective cells and other components.

FIG. 4 is a schematic cross-sectional view of a sensor element 201 according to a modification.

FIG. 5 is a graph showing the relationship between the average height t and the static pressure dependence index α.

### DETAILED DESCRIPTION OF THE INVENTION

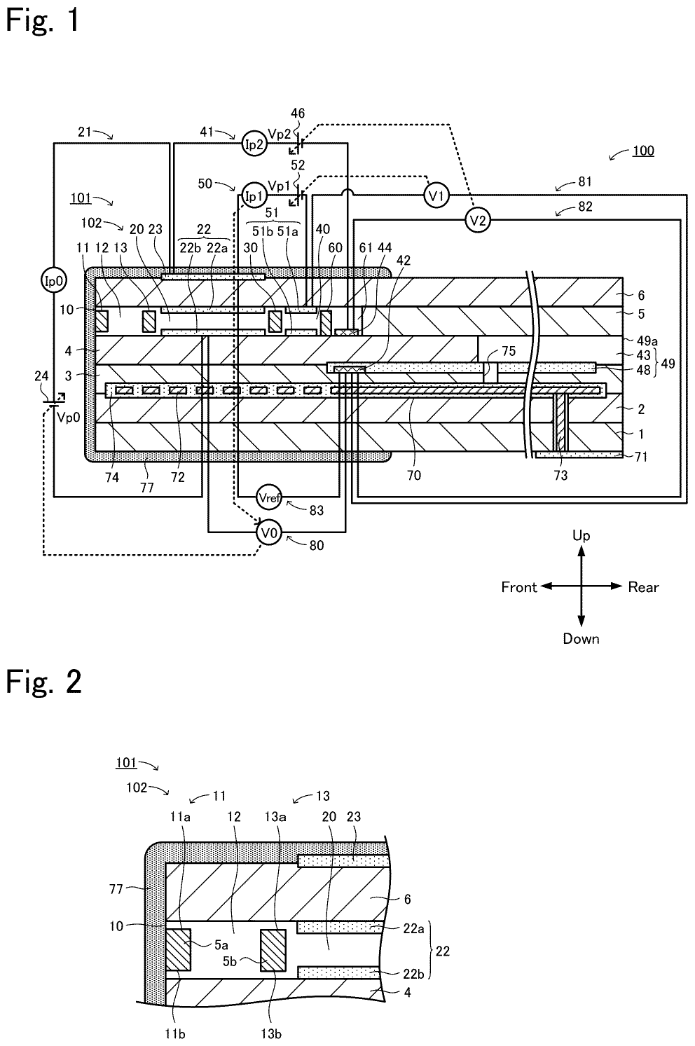

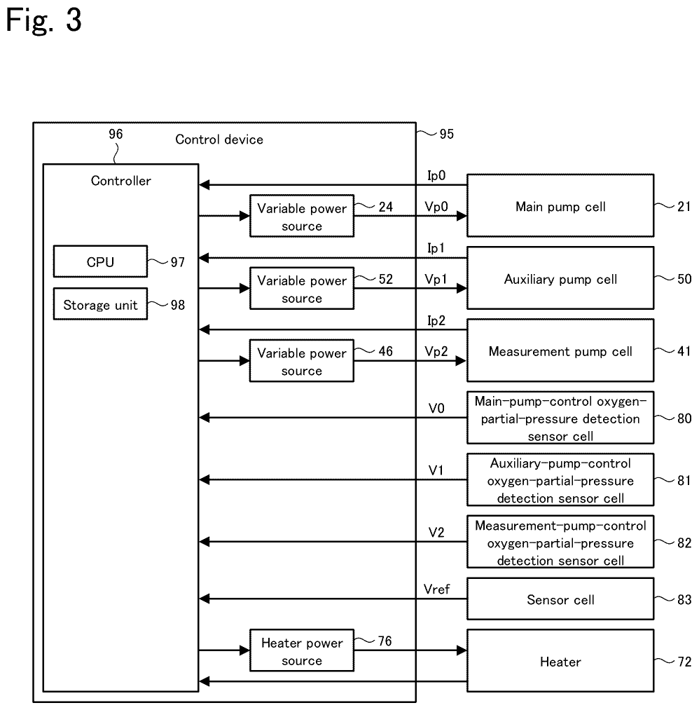

Next, embodiments of the present invention will be described with reference to the drawings. FIG. 1 is a schematic cross-sectional view schematically showing an example configuration of a gas sensor 100 according to an embodiment of the present invention. FIG. 2 is an enlarged view showing an area around the first and second diffusion rate-limiting sections 11 and 13 of the gas sensor 100. FIG. 3 is a block diagram showing the electrical connection relationship between a control device 95, respective cells, and a heater 72. The gas sensor 100 is installed in a pipe, such as an exhaust pipe of an internal combustion engine. The gas sensor 100 detects a concentration of a specific gas, such as NOx or ammonia, in a measurement gas, using exhaust gas from an internal combustion engine as the measurement gas. In the present embodiment, the gas sensor 100 is configured to detect the NOx concentration as the specific gas concentration. The gas sensor 100 includes: a sensor element 101 with an elongated rectangular parallelepiped element body 102; cells 21, 41, 50, and 80 to 83 within the sensor element 101 (i.e. in the element body 102); a heater section 70 provided inside the sensor element 101; and a control device 95, which includes variable power sources 24, 46, and 52, and a heater power source 76, and controls the overall operation of the gas sensor 100.

The sensor element 101 (element body 102) is an element that includes a laminated body in which six layers are stacked in the following order from the bottom in the drawing: a first substrate layer 1, a second substrate layer 2, a third substrate layer 3, a first solid electrolyte layer 4, a spacer layer 5, and a second solid electrolyte layer 6. Each of these layers is composed of an oxygen-ion-conductive solid electrolyte layer, such as zirconia (ZrO₂) or the like. The solid electrolytes forming these six layers are dense and hermetically sealed. The element body 102 is manufactured, for example, by performing predetermined processing and printing of circuit patterns on ceramic green sheets corresponding to the respective layers, laminating the sheets, and then firing the laminated sheets to integrate them into a unified structure.

On the front end side (the left end side in FIG. 1) of the sensor element 101 (element body 102), between the lower surface of the second solid electrolyte layer 6 and the upper surface of the first solid electrolyte layer 4, the following components are formed adjacently and connected in sequence: a gas inlet 10; a first diffusion rate-limiting section 11; a buffer space 12; a second diffusion rate-limiting section 13; a first internal cavity (oxygen concentration adjustment chamber) 20; a third diffusion rate-limiting section 30; a second internal cavity (oxygen concentration adjustment chamber) 40; a fourth diffusion rate-limiting section 60; and a third internal cavity (measurement chamber) 61.

The gas inlet 10, buffer space 12, first internal cavity 20, second internal cavity 40, and third internal cavity 61 are internal spaces within the sensor element 101, formed by hollowing out portions of the spacer layer 5. These spaces are bounded at the top by the lower surface of the second solid electrolyte layer 6, at the bottom by the upper surface of the first solid electrolyte layer 4, and on the sides by the side surfaces of the spacer layer 5.

The first diffusion rate-limiting section 11, the second diffusion rate-limiting section 13, and the third diffusion rate-limiting section 30 are each provided as two horizontally elongated slits (with openings oriented along the longitudinal direction perpendicular to the plane of the drawing). The fourth diffusion rate-limiting section 60 is provided as a single horizontally elongated slit (with openings oriented along the longitudinal direction perpendicular to the plane of the drawing), formed as a gap with the lower surface of the second solid electrolyte layer 6. The area extending from the gas inlet 10 to the third internal cavity 61 is also referred to as the measurement gas flow path.

The sensor element 101 (element body 102) includes a reference gas introduction portion 49, which introduces a reference gas from outside of the sensor element 101 to a reference electrode 42 when measuring the NOx concentration. The reference gas introduction portion 49 comprises a reference gas introduction space 43 and a reference gas introduction layer 48. The reference gas introduction space 43 is an inward space formed from the rear end surface of the sensor element 101. The reference gas introduction space 43 is located between the upper surface of the third substrate layer 3 and the lower surface of the spacer layer 5, and is laterally defined by the side surfaces of the first solid electrolyte layer 4. The reference gas introduction space 43 opens to the rear end surface of the sensor element 101, with this opening serving as an inlet portion 49a of the reference gas introduction portion 49. The reference gas is introduced into the reference gas introduction space 43 through the inlet portion 49a. The reference gas introduction portion 49 introduces the reference gas, which has entered through the inlet portion 49a, to the reference electrode 42 while imparting a predetermined diffusion resistance. In the present embodiment, the reference gas is ambient air.

The reference gas introduction layer 48 is provided between the upper surface of the third substrate layer 3 and the lower surface of the first solid electrolyte layer 4. The reference gas introduction layer 48 is a porous body made of a ceramic material such as alumina or the like. A portion of the upper surface of the reference gas introduction layer 48 is exposed within the reference gas introduction space 43. The reference gas introduction layer 48 is formed so as to cover the reference electrode 42. The reference gas introduction layer 48 allows the reference gas to flow from the reference gas introduction space 43 to the reference electrode 42.

The reference electrode 42 is an electrode formed between the upper surface of the third substrate layer 3 and the first solid electrolyte layer 4, and as described above, the reference gas introduction layer 48, which is connected to the reference gas introduction space 43, is provided around the reference electrode 42. Furthermore, as will be explained later, the reference electrode 42 enables the measurement of the oxygen concentration (oxygen partial pressure) in the first internal cavity 20, the second internal cavity 40, and the third internal cavity 61.

In the measurement gas flow path, the gas inlet 10 is a portion that is open to the external space, allowing the measurement gas to be drawn into the sensor element 101 from the external space. The first diffusion rate-limiting section 11 is a part that imparts a predetermined diffusion resistance to the measurement gas introduced through the gas inlet 10. The first diffusion rate-limiting section 11, as shown in FIG. 2, includes an upper slit 11a and a lower slit 11b. The upper slit 11a is formed as a horizontally elongated slit defined vertically between the upper surface of the partition wall 5a, which is a part of the spacer layer 5, and the lower surface of the second solid electrolyte layer 6. The lower slit 11b is formed as a horizontally elongated slit between the lower surface of the partition wall 5a and the upper surface of the first solid electrolyte layer 4. The partition wall 5a is formed as a portion between the external space and the buffer space 12. The left and right sides of the partition wall 5a are connected to other parts of the spacer layer 5, and there is no gap for the measurement gas to flow between the left and right sides of the partition wall 5a. In the present embodiment, the upper slit 11a and the lower slit lib are formed with the same path length L1 in the front-to-rear direction, and the same width H1 in the lateral direction.

Referring back to FIG. 1, the buffer space 12 is a space provided to guide the measurement gas introduced through the first diffusion rate-limiting section 11 to the second diffusion rate-limiting section 13. The second diffusion rate-limiting section 13 is a portion that imparts a predetermined diffusion resistance to the measurement gas introduced from the buffer space 12 into the first internal cavity 20. The second diffusion rate-limiting section 13, as shown in FIG. 2, includes an upper slit 13a and a lower slit 13b. The upper slit 13a is formed as a horizontally elongated slit between the upper surface of the partition wall 5b, which is a part of the spacer layer 5, and the lower surface of the second solid electrolyte layer 6. The lower slit 13b is formed as a horizontally elongated slit defined vertically between the lower surface of the partition wall 5b and the upper surface of the first solid electrolyte layer 4. The partition wall 5b is formed as a portion between the buffer space 12 and the first internal cavity 20. The left and right sides of the partition wall 5b are connected to other parts of the spacer layer 5, and there is no gap for the measurement gas to flow between the left and right sides of the partition wall 5b. In the present embodiment, the upper slit 13a and the lower slit 13b are formed with the same path length L2 in the front-to-rear direction, and the same width H2 in the lateral direction.

When the measurement gas is introduced from outside the sensor element 101 into the first internal cavity 20, the measurement gas that is abruptly drawn into the sensor element 101 through the gas inlet 10 due to pressure fluctuations in the external space (such as exhaust pulsations in the case where the measurement gas is automobile exhaust gas) is not directly introduced into the first internal cavity 20. Instead, after the pressure fluctuations of the measurement gas are attenuated through the first diffusion rate-limiting section 11, the buffer space 12, and the second diffusion rate-limiting section 13, the measurement gas is introduced into the first internal cavity 20. As a result, the pressure fluctuations of the measurement gas introduced into the first internal cavity 20 become almost negligible. The first internal cavity 20 is provided as a space for adjusting the oxygen partial pressure in the measurement gas introduced through the second diffusion rate-limiting section 13. This oxygen partial pressure is adjusted by the operation of a main pump cell 21.

The main pump cell 21 is an electrochemical pump cell, which is constituted by an inner pump electrode 22 with a ceiling electrode portion 22a provided on nearly the entire lower surface of the second solid electrolyte layer 6 facing the first internal cavity 20, an outer pump electrode 23, which is provided in a manner exposed to the outside of the sensor element 101 in a region of the upper surface of the second solid electrolyte layer 6 corresponding to the ceiling electrode portion 22a, and the second solid electrolyte layer 6, the spacer layer 5, and the first solid electrolyte layer 4, which form the current path between these electrodes.

The inner pump electrode 22 is formed so as to extend across the upper and lower solid electrolyte layers, (namely the second solid electrolyte layer 6 and the first solid electrolyte layer 4), and the spacer layer 5 that provides sidewalls, which together define the first internal cavity 20. Specifically, the ceiling electrode portion 22a is formed on the lower surface of the second solid electrolyte layer 6, which constitutes the ceiling surface of the first internal cavity 20, and a bottom electrode portion 22b is formed on the upper surface of the first solid electrolyte layer 4, which constitutes the bottom surface of the first internal cavity 20. Further, in order to connect the ceiling electrode portion 22a and the bottom electrode portion 22b, side electrode portions (not shown) are formed on the side wall surfaces (inner surfaces) of the spacer layer 5, which constitute both sidewall portions of the first internal cavity 20. The inner pump electrode 22 is disposed in a tunnel-like structure at the region where the side electrode portions are provided.

In the main pump cell 21, a desired voltage Vp0 is applied between the inner pump electrode 22 and the outer pump electrode 23, whereby a pump current Ip0 is caused to flow in a positive direction or a negative direction between the inner pump electrode 22 and the outer pump electrode 23. Thus, the oxygen in the first internal cavity 20 can be pumped out to the external space, or the oxygen in the external space can be pumped into the first internal cavity 20.

Further, in order to detect the oxygen concentration (oxygen partial pressure) in the atmosphere within the first internal cavity 20, an electrochemical sensor cell, that is, a main-pump-control oxygen-partial-pressure detection sensor cell 80, is constituted by the inner pump electrode 22, the second solid electrolyte layer 6, the spacer layer 5, the first solid electrolyte layer 4, the third substrate layer 3, and the reference electrode 42.

By measuring an electromotive force (voltage V0) in the main-pump-control oxygen-partial-pressure detection sensor cell 80, the oxygen concentration (oxygen partial pressure) in the first internal cavity 20 can be determined. Furthermore, by feedback-controlling the voltage Vp0 of the variable power source 24 such that the voltage V0 reaches a target value, the pump current Ip0 is controlled. This configuration allows the oxygen concentration in the first internal cavity 20 to be maintained at a predetermined constant value.

The third diffusion rate-limiting section 30 is a part that imparts a predetermined diffusion resistance to the measurement gas, whose oxygen concentration (oxygen partial pressure) is controlled by the operation of the main pump cell 21 in the first internal cavity 20, and guides the measurement gas into the second internal cavity 40.

The second internal cavity 40 is provided as a space for further adjusting the oxygen partial pressure of the measurement gas, which has already been adjusted for the oxygen concentration (oxygen partial pressure) in the first internal cavity 20, and then being introduced through the third diffusion rate-limiting section 30. This adjustment is carried out by the auxiliary pump cell 50. As a result, the oxygen concentration in the second internal cavity 40 can be maintained at a constant value with high precision, enabling high accuracy NOx concentration measurement in the gas sensor 100.

The auxiliary pump cell 50 is an auxiliary electrochemical pump cell, which is constituted by an auxiliary pump electrode 51 with a ceiling electrode portion 51a provided on nearly the entire lower surface of the second solid electrolyte layer 6 facing the second internal cavity 40, the outer pump electrode 23 (not limited to the outer pump electrode 23, but may be any suitable electrode located outside the sensor element 101), the second solid electrolyte layer 6, the spacer layer 5, and the first solid electrolyte layer 4.

The auxiliary pump electrode 51 is disposed within the second internal cavity 40 in a tunnel-like structure similar to that of the inner pump electrode 22 disposed in the first internal cavity 20 described above. Specifically, the ceiling electrode portion 51a is formed on the second solid electrolyte layer 6, which constitutes the ceiling surface of the second internal cavity 40, and a bottom electrode portion 51b is formed on the first solid electrolyte layer 4, which constitutes the bottom surface of the second internal cavity 40. Further, side electrode portions (not shown), which connect the ceiling electrode portion 51a and the bottom electrode portion 51b, are formed on the inner side surfaces of the spacer layer 5, which constitute both sidewall portions of the second internal cavity 40. Thus, the auxiliary pump electrode 51 is formed in a tunnel-like structure.

In the auxiliary pump cell 50, a desired voltage Vp1 is applied between the auxiliary pump electrode 51 and the outer pump electrode 23. Thus, the oxygen in the atmosphere within the second internal cavity 40 can be pumped out to the external space, or the oxygen can be pumped into the second internal cavity 40 from the external space.

Further, in order to control the oxygen partial pressure in the atmosphere within the second internal cavity 40, an electrochemical sensor cell, that is, an auxiliary-pump-control oxygen-partial-pressure detection sensor cell 81, is constituted by the auxiliary pump electrode 51, the reference electrode 42, the second solid electrolyte layer 6, the spacer layer 5, the first solid electrolyte layer 4, and the third substrate layer 3.

The auxiliary pump cell 50 performs pumping via the variable power source 52, which is voltage-controlled based on an electromotive force (voltage V1) detected by the auxiliary-pump-control oxygen-partial-pressure detection sensor cell 81. As a result, the oxygen partial pressure in the atmosphere within the second internal cavity 40 is controlled to a low level at which it does not substantially affect the measurement of NOx.

In addition, a pump current Ip1 is also used for controlling the electromotive force of the main-pump-control oxygen-partial-pressure detection sensor cell 80. Specifically, the pump current Ip1 is input as a control signal to the main-pump-control oxygen-partial-pressure detection sensor cell 80, and by controlling the above-mentioned target value of the voltage V0, the oxygen partial pressure gradient in the measurement gas introduced from the third diffusion rate-limiting section 30 into the second internal cavity 40 is maintained constant at all times. When used as a NOx sensor, the oxygen concentration in the second internal cavity 40 is maintained at a constant value of approximately 0.001 ppm by the operation of the main pump cell 21 and the auxiliary pump cell 50.

The fourth diffusion rate-limiting section 60 is a part that imparts a predetermined diffusion resistance to the measurement gas, whose oxygen concentration (oxygen partial pressure) is controlled by the operation of the auxiliary pump cell 50 in the second internal cavity 40, and guides the measurement gas into the third internal cavity 61. The fourth diffusion rate-limiting section 60 serves to limit the amount of NOx flowing into the third internal cavity 61.

The third internal cavity 61 is provided as a space for processing the measurement of the nitrogen oxide (NOx) concentration in the measurement gas, which has already been adjusted for the oxygen concentration (oxygen partial pressure) in the second internal cavity 40, and then being introduced through the fourth diffusion rate-limiting section 60. The measurement of the NOx concentration is primarily carried out by the operation of the measurement pump cell 41 in the third internal cavity 61.

The measurement pump cell 41 measures the NOx concentration in the measurement gas within the third internal cavity 61. This measurement pump cell 41 is an electrochemical pump cell, which is constituted by a measurement electrode 44 provided on the upper surface of the first solid electrolyte layer 4 facing the third internal cavity 61, the outer pump electrode 23, the second solid electrolyte layer 6, the spacer layer 5, and the first solid electrolyte layer 4. The measurement electrode 44 also functions as a NOx reduction catalyst, reducing NOx present in the atmosphere within the third internal cavity 61.

In the measurement pump cell 41, oxygen generated by the decomposition of nitrogen oxides in the atmosphere around the measurement electrode 44 is pumped out, and the amount of oxygen generated can be detected as a pump current Ip2.

Further, in order to detect the oxygen partial pressure around the measurement electrode 44, an electrochemical sensor cell, that is, a measurement-pump-control oxygen-partial-pressure detection sensor cell 82, is constituted by the first solid electrolyte layer 4, the third substrate layer 3, the measurement electrode 44, and the reference electrode 42. Based on the electromotive force (voltage V2) detected by the measurement-pump-control oxygen-partial-pressure detection sensor cell 82, the variable power source 46 is controlled.

The measurement gas introduced into the second internal cavity 40 reaches the measurement electrode 44 in the third internal cavity 61 through the fourth diffusion rate-limiting section 60, under controlled conditions of oxygen partial pressure. Nitrogen oxides present in the measurement gas around the measurement electrode 44 are reduced (2NO→N₂+O₂), thereby generating oxygen. This generated oxygen is then pumped by the measurement pump cell 41. During this process, the voltage Vp2 of the variable power source 46 is controlled to maintain the voltage V2, detected by the measurement-pump-control oxygen-partial-pressure detection sensor cell 82, at a constant (target) value. Since the amount of oxygen generated around the measurement electrode 44 is proportional to the concentration of nitrogen oxides in the measurement gas, the nitrogen oxide concentration in the measurement gas is determined based on the pump current Ip2 of the measurement pump cell 41.

Further, by combining the measurement electrode 44, the first solid electrolyte layer 4, the third substrate layer 3, and the reference electrode 42, an oxygen-partial-pressure detection device can be configured as an electrochemical sensor cell. In this configuration, it is possible to detect an electromotive force corresponding to the difference between the amount of oxygen generated by the reduction of NOx components in the atmosphere around the measurement electrode 44 and the amount of oxygen in a reference atmosphere. This enables the determination of the concentration of NOx components in the measurement gas.

Furthermore, an electrochemical sensor cell 83 is constituted by the second solid electrolyte layer 6, the spacer layer 5, the first solid electrolyte layer 4, the third substrate layer 3, the outer pump electrode 23, and the reference electrode 42. The electromotive force (voltage Vref) detected by this sensor cell 83 enables the detection of the oxygen partial pressure in the measurement gas outside the sensor.

In the gas sensor 100 with such a configuration, the measurement gas, in which the oxygen partial pressure is always maintained at a constant low value (a value that does not substantially affect NOx measurement), is supplied to the measurement pump cell 41 by operating the main pump cell 21 and the auxiliary pump cell 50. Accordingly, the NOx concentration in the measurement gas can be determined based on the pump current Ip2, which flows as oxygen generated by the reduction of NOx is pumped out from the measurement pump cell 41. The amount of this current is substantially proportional to the NOx concentration in the measurement gas.

Each of the electrodes 22, 23, 42, 44, and 51 will be described. The inner pump electrode 22, the auxiliary pump electrode 51, and the measurement electrode 44 each contains a first type of noble metal with catalytic activity. Examples of the first type of noble metal include, but are not limited to, at least one selected from Pt, Rh, Ir, Ru, and Pd. The outer pump electrode 23 and the reference electrode 42 also contain the first type of noble metal. The inner pump electrode 22 and the auxiliary pump electrode 51 further contain second type of noble metal for suppressing the catalytic activity of the first type of noble metal with respect to the specific gas (NOx). As a result, the reducing capability of the inner pump electrode 22 and the auxiliary pump electrode 51 with respect to NOx components in the measurement gas is reduced. An example of the second type of noble metal is Au. The measurement electrode 44 does not contain the second type of noble metal. Accordingly, the reducing capability of the measurement electrode 44 with respect to NOx components in the measurement gas is greater than that of the inner pump electrode 22 and the auxiliary pump electrode 51. Preferably, the outer pump electrode 23 and the reference electrode 42 also do not contain the second type of noble metal. Each of the electrodes 22, 23, 42, 44, and 51 is preferably a cermet containing a noble metal and an oxide with oxygen ion conductivity (e.g. ZrO₂). Each of the electrodes 22, 23, 42, 44, and 51 is also preferably porous body. In the present embodiment, the inner pump electrode 22 and the auxiliary pump electrode 51 are each porous cermet electrodes composed of Pt containing 1% Au and ZrO₂. The outer pump electrode 23, the reference electrode 42, and the measurement electrode 44 are each porous cermet electrodes composed of Pt and ZrO₂.

The sensor element 101 includes a heater section 70 that performs temperature regulation by heating and maintaining the temperature of the sensor element 101, in order to enhance the oxygen-ion-conductivity of the solid electrolyte. The heater section 70 includes a heater connector electrode 71, a heater 72, a through hole 73, a heater insulating layer 74, and a pressure relief hole 75.

The heater connector electrode 71 is an electrode formed in such a manner as to be in contact with the lower surface of the first substrate layer 1. By connecting the heater connector electrode 71 to a heater power source 76 (see FIG. 2), power can be supplied from the heater power source 76 to the heater section 70.

The heater 72 is an electrical resistor formed in such a manner as to be sandwiched between the second substrate layer 2 and the third substrate layer 3 from above and below. The heater 72 is connected to the heater connector electrode 71 via the through hole 73, and generates heat when power is supplied from the heater power source 76 through the heater connector electrode 71, thereby heating and maintaining the temperature of the solid electrolyte forming the sensor element 101.

The heater 72 is also embedded across the entire region from the first internal cavity 20 to the third internal cavity 61, making it possible to adjust the temperature of the entire sensor element 101 to a level at which the solid electrolyte is activated.

The heater insulating layer 74 is an insulating layer formed of an insulator such as alumina and provided on the upper and lower surfaces of the heater 72. The heater insulating layer 74 is formed for the purpose of providing electrical insulation between the second substrate layer 2 and the heater 72, as well as between the third substrate layer 3 and the heater 72.

The pressure relief hole 75 is a portion that penetrates through the third substrate layer 3 and the reference gas introduction layer 48, and is formed so as to communicate with the reference gas introduction space 43. The pressure relief hole 75 is formed for the purpose of relieving an increase in internal pressure caused by a rise in temperature within the heater insulating layer 74.

As shown in FIG. 3, the control device 95 includes the variable power sources 24, 46, and 52 described above, the heater power source 76 also described above, and a control unit 96. The control unit 96 is a microprocessor including a CPU 97 and a storage unit 98. The storage unit 98 is a rewritable non-volatile memory, and is capable of storing, for example, various programs and various kinds of data. The control unit 96 inputs the voltage V0 from the main-pump-control oxygen-partial-pressure detection sensor cell 80, the voltage V1 from the auxiliary-pump-control oxygen-partial-pressure detection sensor cell 81, the voltage V2 from the measurement-pump-control oxygen-partial-pressure detection sensor cell 82, the voltage Vref from the sensor cell 83, the pump current Ip0 flowing through the main pump cell 21, the pump current Ip1 flowing through the auxiliary pump cell 50, and the pump current Ip2 flowing through the measurement pump cell 41. In addition, the control unit 96 controls the voltages Vp0, Vp1, and Vp2 output from the variable power sources 24, 46, and 52, respectively, by outputting control signals to the variable power sources 24, 46, and 52. Through this control, the main pump cell 21, the measurement pump cell 41, and the auxiliary pump cell 50 are controlled. The control unit 96 also controls the power supplied from the heater power source 76 to the heater 72 by outputting a control signal to the heater power source 76. The storage unit 98 also stores target values V0*, V1*, and V2*, which will be described later. The CPU 97 of the control unit 96 performs control of the respective cells 21, 41, and 50 with reference to the target values V0*, V1*, and V2*.

The control unit 96 performs auxiliary pump control processing to control the auxiliary pump cell 50 such that the oxygen concentration in the second internal cavity 40 reaches a target concentration. Specifically, the control unit 96 controls the auxiliary pump cell 50 by feedback-controlling the voltage Vp1 of the variable power source 52 such that the voltage V1 reaches a constant value (referred to as the target value V1*). The target value V1* is set as a value such that the oxygen concentration in the second internal cavity 40 is at a predetermined low concentration that does not substantially affect the NOx measurement.

The control unit 96 performs main pump control processing to control the main pump cell 21 such that the pump current Ip1, which flows when the auxiliary pump cell 50 adjusts the oxygen concentration in the second internal cavity 40 during the auxiliary pump control processing, reaches a target current (referred to as a target value Ip1*). Specifically, the control unit 96 sets (via feedback control) a target value for the voltage V0 (referred to as the target value V0*) based on the pump current Ip1, such that the pump current Ip1 flowing through the voltage Vp1 reaches the constant target value Ip1*. The control unit 96 then feedback-controls the voltage Vp0 of the variable power source 24 such that the voltage V0 reaches the target value V0* (i.e. such that the oxygen concentration in the first internal cavity 20 reaches the target concentration). Through this main pump control processing, the gradient of the oxygen partial pressure in the measurement gas, which is introduced from the third diffusion rate-limiting section 30 into the second internal cavity 40, is maintained at a constant value at all times. The target value V0* is set such that the oxygen concentration in the first internal cavity 20 is greater than 0% but is at a low concentration. Additionally, the pump current Ip0 flowing during this main pump control processing changes in accordance with the oxygen concentration of the measurement gas (i.e. the measurement gas around the sensor element 101) as it flows from the gas inlet 10 into the measurement gas flow path. As a result, the control unit 96 can also detect the oxygen concentration in the measurement gas based on the pump current Ip0.

The main pump control processing and the auxiliary pump control processing described above are collectively referred to as an adjustment pump control processing. Additionally, the first internal cavity 20 and the second internal cavity 40 are collectively referred to as an oxygen concentration adjustment chamber. The main pump cell 21 and the auxiliary pump cell 50 are collectively referred to as adjustment pump cells. By performing the adjustment pump control processing, the control unit 96 causes the adjustment pump cells to adjust the oxygen concentration in the oxygen concentration adjustment chamber.

Furthermore, the control unit 96 performs measurement pump control processing to control the measurement pump cell 41 such that the voltage V2 reaches a constant value (referred to as the target value V2*) (that is, the oxygen concentration in the third internal cavity 61 reaches a predetermined low level). Specifically, the control unit 96 controls the measurement pump cell 41 by feedback-controlling the voltage Vp2 of the variable power source 46 such that the voltage V2 reaches the target value V2*. Through this measurement pump control processing, oxygen is pumped out of the third internal cavity 61.

Through the performance of the measurement pump control processing, oxygen is pumped out of the third internal cavity 61, such that the oxygen generated by the reduction of NOx in the measurement gas within the third internal cavity 61 is reduced to substantially zero. The control unit 96 then acquires the pump current Ip2 as a detection value corresponding to the oxygen generated in the third internal cavity 61 originating from the specific gas (in this case, NOx), and calculates the NOx concentration in the measurement gas based on this pump current Ip2.

The storage unit 98 stores a formula (such as a linear function or quadratic equation) or a map that defines the correspondence relationship between the pump current Ip2 and the NOx concentration. This formula or map can be determined in advance through experiments.

The control unit 96 performs heater control processing by outputting a control signal to the heater power source 76 such that the temperature of the heater 72 reaches to a target temperature (e.g. 800° C.). Here, the temperature of the heater 72 can be expressed as a linear function of the resistance value of the heater 72. In the heater control processing, the control unit 96 calculates the resistance value of the heater 72, which is a value that can be regarded as the temperature of the heater 72 (a value that can be converted into temperature), and feedback-controls the heater power source 76 such that the calculated resistance value reaches to a target resistance value (a value corresponding to the target temperature). The control unit 96 can acquire the voltage of the heater 72 and the current flowing through the heater 72, then calculate the resistance of the heater 72 based on the acquired voltage and current. The control unit 96 may use a three-wire or four-wire method to calculate the resistance, for example. The heater power source 76 adjusts the power supplied to the heater 72 by changing the voltage applied to the heater 72 based on a control signal from the control unit 96 when energizing the heater 72.

In addition, the control device 95, which includes the variable power sources 24, 46, 52, and the heater power source 76 shown in FIG. 3, is actually connected to the electrodes inside the sensor element 101 through unillustrated lead wires formed within the sensor element 101 and unillustrated connector electrodes formed at the rear end of the sensor element 101 (only the heater connector electrode 71 shown in FIG. 1).

Additionally, the element body 102 of the sensor element 101 is covered at the front end portion with a porous protective layer 77, as shown in FIG. 1. This porous protective layer 77 covers portions of both the upper and lower surfaces of the element body 102. Although not shown in the drawing, the porous protective layer 77 also covers portions of the left and right surfaces of the element body 102. The porous protective layer 77 also covers the front surface of the element body 102. The porous protective layer 77 also covers the outer pump electrode 23. The porous protective layer 77 also covers the gas inlet 10. Since the porous protective layer 77 is a porous body, the measurement gas can flow through the inside of the porous protective layer 77 and reach the outer pump electrode 23 and the gas inlet 10. The porous protective layer 77 covers a portion of the element body 102, thereby protecting that part. The porous protective layer 77 functions to prevent the formation of cracks in the element body 102, which could be caused by the adhesion of moisture or other substances from the measurement gas. Additionally, the porous protective layer 77 serves to prevent contaminants, such as oil components contained in the measurement gas, from adhering to the outer pump electrode 23, thereby reducing the deterioration of the outer pump electrode 23. The porous protective layer 77 is a porous body made of a ceramic material, such as alumina. The porosity of the porous protective layer 77 is not particularly limited, but generally ranges from 10% to 60%. The thickness t of the porous protective layer 77 (the length in the front-rear direction of the portion that covers the front side of the element body 102) is preferably in the range of approximately 300 μm to 700 μm.

The porosity of the porous protective layer 77 is derived as follows, using the image (SEM image) obtained by observing with a scanning electron microscope (SEM). First, the object to be measured is cut such that its cross-section is used as the observation surface. The cut surface is then resin-embedded and polished to prepare a sample for observation. Next, SEM photographs (secondary electron image, acceleration voltage of 15 kV, magnification of 1000 times; if the 1000 times magnification is unsuitable, a magnification greater than 1000 times but less than or equal to 5000 times is used) are taken to obtain the SEM image of the object to be measured. The obtained image is then analyzed using image analysis, and a threshold is determined using discriminant analysis (Otsu's binarization) based on the luminance distribution of the pixel luminance data in the image. Based on the determined threshold, each pixel in the image is binarized into object and pore portions, and the areas of the object portion and the pore portion are calculated. The ratio of the area of the pore portion to the total area (the sum of the object portion and pore portion) is derived as the porosity (unit: %).

Next, an example of the manufacturing method for the sensor element 101 of the gas sensor 100 will be described below. First, six pieces of unfired ceramic green sheets, containing an oxygen-ion-conductive solid electrolyte such as zirconia, are prepared as ceramic components. These green sheets are pre-formed with several holes, such as sheet holes used for positioning during printing or lamination, and necessary through-holes. Additionally, the green sheet for the spacer layer 5 is pre-punched to form a space that will serve as the gas flow path for the measurement gas. Similarly, the green sheet for the first solid electrolyte layer 4 is pre-provided with a space that will serve as the reference gas introduction space 43. Then, for each of the first substrate layer 1, second substrate layer 2, third substrate layer 3, first solid electrolyte layer 4, spacer layer 5, and second solid electrolyte layer 6, pattern printing and drying processes are performed to form various patterns on each ceramic green sheet. The patterns to be formed include, for example, the electrodes described above, lead wires connected to each electrode, the reference gas introduction layer 48, and the heater section 70. The pattern printing process is performed by applying a pattern-forming paste, which is prepared according to the required characteristics of each component, onto the green sheets using a known screen printing technique. The drying process is also carried out using known drying methods. After the pattern printing and drying process are completed, the printing and drying processes for adhesive paste to laminate and bond the green sheets corresponding to each layer are carried out. The green sheets with adhesive paste are then aligned using positioning holes and laminated in a predetermined order. The lamination process is carried out by applying predetermined temperature and pressure conditions to bond the sheets into a single laminated body. The laminated body obtained in this manner includes multiple element bodies 102. This laminated body is cut into pieces to the size of the element body 102. Then, the cut laminated body is fired at a predetermined firing temperature to obtain the element body 102.

Next, the porous protective layer 77 is formed on the element body 102 to obtain the sensor element 101. The porous protective layer 77 can be formed using at least one of the following methods: plasma spraying, screen printing, gel casting, or dipping. When the porous protective layer 77 is formed by methods involving firing, such as screen printing or dipping, the porous protective layer 77 may be applied to the element body 102 before firing, and both the element body 102 and the porous protective layer 77 may be fired together to obtain the sensor element 101. After obtaining the sensor element 101 in this manner, it can be housed in a predetermined housing and incorporated into a gas sensor body (not shown), thereby resulting in the gas sensor 100.

Here, the fourth diffusion rate-limiting section 60 can be formed, for example, as follows. First, during the pattern printing process described above, a vanishing material (e.g. theobromine) that is eliminated by firing is applied to the upper surface of the portion of the green sheet that will form the partition wall of the spacer layer 5. As a result, during the subsequent firing process, the sacrificial material disappears, and a gap (a horizontally elongated slit) is formed between the upper surface of the partition wall of the spacer layer 5 and the lower surface of the second solid electrolyte layer 6, thereby forming the fourth diffusion rate-limiting section 60. The sacrificial material may also be applied not only to the upper surface of the partition wall portion but also to the portion of the lower surface of the green sheet that will form the second solid electrolyte layer 6 and that faces the partition wall. Furthermore, the vertical height of the slit constituting the fourth diffusion rate-limiting section 60 may be adjusted by controlling the thickness of the applied sacrificial material. The first through third diffusion rate-limiting sections 11, 13, and 30 may also be formed in a similar manner, except that the sacrificial material is applied to both the upper and lower surfaces of the spacer layer 5. It should be noted that such a method for forming diffusion rate-limiting sections is known and is disclosed, for example, in Japanese Patent No. 4911910.

Next, an example of the use of the gas sensor 100 will be described. It is assumed that the CPU 97 of the control device 95 is performing control of the pump cells 21, 41, and 50 (adjustment pump control processing and measurement pump control processing), and is acquiring the respective voltages V0, V1, V2, and Vref from the sensor cells 80 to 83. In this state, when the measurement gas is introduced into the element body 102 through the gas inlet 10, the measurement gas first flows, in this order, through the first diffusion rate-limiting section 11, the buffer space 12, and the second diffusion rate-limiting section 13, and reaches the first internal cavity 20. Subsequently, in the first internal cavity 20 and the second internal cavity 40, the oxygen concentration of the measurement gas is adjusted by the main pump cell 21 and the auxiliary pump cell 50. The measurement gas after adjustment then reaches the third internal cavity 61. Thereafter, the CPU 97 acquires the pump current Ip2 and detects the NOx concentration in the measurement gas based on the acquired pump current Ip2.

In the present embodiment, the sensor element 101 is configured such that the average height t of the first and second diffusion rate-limiting sections 11 and 13 is 0.0035 mm or greater. Preferably, the sensor element 101 is configured such that the average height t of the first and second diffusion rate-limiting sections 11 and 13 is 0.0090 mm or greater. Also, preferably, the sensor element 101 is configured such that the average height t of the first and second diffusion rate-limiting sections 11 and 13 is 0.0250 mm or less. The average height t of the first and second diffusion rate-limiting sections 11 and 13 corresponds to the average of the heights t1 and t2 of the diffusion rate-limiting sections 11 and 13. The height t1 of the first diffusion rate-limiting section 11 corresponds to the sum of the height t11 of the upper slit 11a and the height t12 of the lower slit 11b of the first diffusion rate-limiting section 11. The height t2 of the second diffusion rate-limiting section 13 corresponds to the sum of the height t21 of the upper slit 13a and the height t22 of the lower slit 13b of the second diffusion rate-limiting section 13.

The average height t of the first and second diffusion rate-limiting sections 11 and 13 is obtained using Equation (A) based on the following parameters: the path lengths L1 and L2 [cm] of the first and second diffusion rate-limiting sections 11 and 13; the widths H1 and H2 [cm] of the first and second diffusion rate-limiting sections 11 and 13; the limiting current Ip [A] of the main pump cell 21; the Faraday constant F [A·sec/mol]; the diffusion coefficient D [cm²/see] of oxygen; the gas constant R [cm³·atm/mol·K]; the temperature T [K] of the inner pump electrode 22; the oxygen partial pressure Poe [atm] in the measurement gas; and the oxygen partial pressure Pod [atm] in the first internal cavity 20. The path lengths L1 and L2 of the first and second diffusion rate-limiting sections 11 and 13 correspond to the path lengths, that is, the lengths in the forward-backward direction, of the upper slits 11a, 13a and the lower slits 11b, 13b, respectively. The widths H1 and H2 of the first and second diffusion rate-limiting sections 11 and 13 correspond to the widths, that is, the lengths in the left-right direction, of the upper slits 11a, 13a and the lower slits 11b, 13b, respectively. When the upper slits 11a, 13a and the lower slits 11b, 13b of the first and second diffusion rate-limiting sections 11 and 13 are considered as a single diffusion rate-limiting section as a whole, the height t of this diffusion rate-limiting section can be obtained using Equation (2) based on the path length L and the width H of the diffusion rate-limiting section, the diffusion coefficient D of oxygen, the gas constant R, the temperature T of the inner pump electrode 22, the oxygen partial pressure Poe in the measurement gas, and the oxygen partial pressure Pod in the first internal cavity 20. Equation (1) corresponds to the version of Equation (2) where the term “L/H” on the right-hand side is replaced with “L1/H1+L2/H2”.

$$t = ( L 1 / H 1 + L 2 / H 2 ) \times Ip \times 1 / ( 4 \times F \times D / ( R \times T ) ) \times 1 / ( Poe - Pod ) \times 10 \qquad ( 1 )$$

$$t = L / H \times Ip \times 1 / ( 4 \times F \times D / ( R \times T ) ) \times 1 / ( Poe - Pod ) \times 10 \qquad ( 2 )$$

In Equation (1), the path length L1 of the first diffusion rate-limiting section 11 is set to 300 μm in the present embodiment, and the path length L2 of the second diffusion rate-limiting section 13 is set to 500 μm in the present embodiment. The widths H1 and H2 of the first and second diffusion rate-limiting sections 11 and 13 are each within the range of 1.0 mm to 2.5 mm in the present embodiment, respectively. The Faraday constant F is set to 96490 [A·sec/mol] in the present embodiment. The diffusion coefficient D of oxygen is set to 1.6 [cm²/sec] in the present embodiment. The gas constant R is set to 82.05 [cm³·atm/mol·K] in the present embodiment.

In Equation (1), the limiting current Ip of the main pump cell 21, in the present embodiment, is the limiting current value of the pump current Ip0 obtained when oxygen is pumped out from the around of the inner pump electrode 22 to the around of the outer pump electrode 23, under a condition in which the gas inlet 10 of the sensor element 101 is exposed to an ambient gas in which the base gas is nitrogen, the oxygen concentration is 21%, and the pressure is 1 atm.

The limiting current Ip of the main pump cell 21 can be measured, for example, as follows. First, the gas inlet 10 of the sensor element 101 is exposed to an ambient gas in which the base gas is nitrogen, the oxygen concentration is 21%, and the pressure is 1 atm. For example, the gas sensor 100 including the sensor element 101 is installed in a pipe such that the tip side portion of the sensor element 101 protrudes into the pipe, and the ambient gas is flowed through the pipe, whereby the gas inlet 10 of the sensor element 101 is exposed to this ambient gas. Although the oxygen concentration around the reference gas introduction portion 49 does not significantly affect the measured value of the limiting current Ip, the reference gas introduction portion 49 is exposed to the atmosphere. Subsequently, the heater 72 is energized to heat the sensor element 101 to a predetermined operating temperature Tset (e.g. 800° C.). At this time, none of the variable power sources 24, 46, and 52 apply voltage. After the temperature of the sensor element 101 has stabilized, a voltage Vp0 is applied between the inner pump electrode 22 and the outer pump electrode 23 by the variable power source 24 such that oxygen is pumped out from around the inner pump electrode 22 to around the outer pump electrode 23. During this operation, the pump current Ip0 (oxygen pumping out current) flowing between the electrodes 22 and 23 is then measured. The voltage Vp0 is a direct current (DC) voltage. Then, as the voltage Vp0 is gradually increased, the pump current Ip0 also gradually increases. However, eventually, even if the voltage Vp0 is further increased, the pump current Ip0 does not increase and reaches its upper limit. The pump current Ip0 at this point is measured as the limiting current Ip. The flow rate of the gas reaching the first internal cavity 20, i.e., around the inner pump electrode 22, through the porous protective layer 77, the gas inlet 10, the first diffusion rate-limiting section 11, the buffer space 12, and the second diffusion rate-limiting section 13 from the outside, depends on the diffusion resistance of the predetermined portion of the gas path from the outside to the inner pump electrode 22. The diffusion resistance of the predetermined portion has a negative correlation with the limiting current Ip of the main pump cell 21. Furthermore, the diffusion resistance of the predetermined portion (the limiting current Ip of the main pump cell 21) is particularly influenced by the diffusion resistance of the first and second diffusion rate-limiting sections 11 and 13 upstream of the first internal cavity 20. The diffusion resistance of the first and second diffusion rate-limiting sections 11 and 13 is affected by the shape of the upper slits 11a and 13a and the lower slits 11b and 13b of the first and second diffusion rate-limiting sections 11 and 13, specifically, the path lengths L1 and L2, the widths H1 and H2, and the heights t11, t12, t21, and t22. Therefore, the diffusion rate-limiting sections used for calculating the average height t are the first and second diffusion rate-limiting sections 11 and 13, out of the first to fourth diffusion rate-limiting sections 11, 13, 40, and 60.

In Equation (1), the temperature T of the inner pump electrode 22 is an estimated value of the temperature of the inner pump electrode 22 when the limiting current Ip of the main pump cell 21 are measured, and when the adjustment pump control processing described above (the auxiliary pump control processing using the target value V1* described above, and the main pump control processing) is performed. In the present embodiment, the temperature T is measured as follows. First, a temperature measurement sample is produced in which a resistance element for temperature measurement is disposed at the position of the inner pump electrode 22 of the sensor element 101. Subsequently, the heater 72 of the temperature measurement sample is energized to heat the sensor element 101 to the operating temperature Tset described above, and when the temperature of the sensor element 101 becomes stable, the temperature calculated from the resistance value of the resistance element is measured as the temperature T. Alternatively, the temperature T may also be measured by a method such as thermographic measurement, once the heater 72 of the sensor element 101 is energized to heat the sensor element 101 to the predetermined operating temperature Tset and the temperature of the sensor element 101 becomes stable. In the present embodiment, the temperature T of the inner pump electrode 22 is set to 1123 K.

In Equation (1), the oxygen partial pressure Poe in the measurement gas is an estimated value of the oxygen partial pressure in the measurement gas when measuring the limiting current Ip of the main pump cell 21. In the present embodiment, since an ambient gas in which the base gas is nitrogen, the oxygen concentration is 21%, and the pressure is 1 atm is used, the oxygen partial pressure Poe is a partial pressure corresponding to this atmospheric condition. The oxygen partial pressure Pod in the first internal cavity 20 is the oxygen partial pressure corresponding to the oxygen concentration in the first internal cavity 20, which is adjusted by performing the adjustment pump control processing (the auxiliary pump control processing using the target value V1* described above, and the main pump control processing). For example, the oxygen partial pressure Pod can be obtained by acquiring the voltage V0 and the oxygen partial pressure Podr of the reference gas (air) when the adjustment pump control processing is performed, and applying these to a predetermined correspondence relationship between the voltage V0, the oxygen partial pressure Podr, and the oxygen partial pressure Pod. In the present embodiment, the value obtained by subtracting the oxygen partial pressure Pod in the first internal cavity 20 from the oxygen partial pressure Poe in the measurement gas (Poe−Pod) is set to 0.209999 atm.

In the gas sensor 100, as the average height t of the first and second diffusion rate-limiting sections 11 and 13 decreases, the diffusion resistance at a predetermined portion increases. As a result, the dependence of the pump current Ip0 of the main pump cell 21 (i.e., the most upstream pump cell among the main and auxiliary pump cells 21 and 50) on the pressure of the measurement gas becomes higher. In the present embodiment, the sensor element 101 is configured such that the average height t of the first and second diffusion rate-limiting sections 11 and 13 is 0.0035 mm or greater. This configuration suppresses the diffusion resistance from becoming excessively large and prevents an excessive increase in the static pressure dependence of the pump current Ip0. The inventors have confirmed this effect through experiments and analysis. Accordingly, a sensor element 101 capable of suppressing the excessive static pressure dependence of the pump current Ip0 can be provided. In other words, by designing the upper slits 11a, 13a and the lower slits 11b, 13b of the first and second diffusion rate-limiting sections 11 and 13, including their path lengths (L1, L2), widths (H1, H2), and heights (t11, t12, t21, t22), such that the average height t is 0.0035 mm or greater, the static pressure dependence of the pump current Ip0 can be effectively suppressed. Furthermore, when the sensor element 101 is configured such that the average height t of the first and second diffusion rate-limiting sections 11 and 13 is 0.0090 mm or greater, the excessive increase in the static pressure dependence of the pump current Ip0 can be further effectively suppressed. In addition, as the average height t of the first and second diffusion rate-limiting sections 11 and 13 increases, the diffusion resistance of the predetermined portion that serves as the gas path from the outside up to the inner pump electrode 22 decreases, and a greater amount of oxygen is introduced around the inner pump electrode 22 from the outside. Accordingly, the deterioration rate of the inner pump electrode 22 tends to increase. The deterioration of the inner pump electrode 22 may occur, for example, when the first type of noble metal (e.g. Pt) contained in the inner pump electrode 22 oxidizes and sublimates, thereby decreasing the catalytic activity with respect to a specific gas (NOx), which in turn lowers the oxygen pumping capability of the main pump cell 21. The deterioration of the inner pump electrode 22 may also occur when the second type of noble metal (e.g. Au) contained in the inner pump electrode 22 evaporates. This may cause the specific gas (NOx) to be decomposed at the inner pump electrode 22. Additionally, the second type of noble metal may adhere to the measurement electrode 44, reducing its catalytic activity with respect to the specific gas and resulting in decreased NOx detection accuracy. In contrast, by configuring the sensor element 101 such that the average height t of the first and second diffusion rate-limiting sections 11 and 13 is 0.0250 mm or less, it is possible to suppress an excessive increase in the deterioration rate of the inner pump electrode 22. The inventors have confirmed this through experiments and analysis. As a result, it is possible to provide a sensor element 101 capable of suppressing the excessive increase in the deterioration rate of the inner pump electrode 22.

In the present embodiment, as described above, the element body 102 is manufactured by firing the six-layer laminated body to form an integrated structure. As a result, the central portion of the upper slits 11a and 13a and the lower slits 11b and 13b may bulge or dent in the width direction relative to their respective end portions. Accordingly, the heights t11 and t12 of the upper slits 11a and 13a and the heights t21 and t22 of the lower slits 11b and 13b often vary depending on the position in the width direction, making it difficult to accurately measure the heights t11, t12, t21, and t22. Consequently, it is also difficult to calculate the average height t of the first and second diffusion rate-limiting sections 11 and 13 based on those measured values. In contrast, by using Equation (1), it is possible to calculate the average height t of the first and second diffusion rate-limiting sections 11 and 13.

Here, the correspondence relationship between the elements according to the present embodiment and the elements according to the present invention will be clarified. Each of the first substrate layer 1, the second substrate layer 2, the third substrate layer 3, the first solid electrolyte layer 4, the spacer layer 5, and the second solid electrolyte layer 6 according to the present embodiment corresponds to the solid electrolyte layer according to the present invention. The element body 102 corresponds to the element body. The first and second internal cavities 20 and 40 correspond to the oxygen concentration adjustment chambers. The inner pump electrode 22 and auxiliary pump electrode 51 correspond to the inner electrodes. The main pump cell 21 and auxiliary pump cell 50 correspond to the adjustment pump cells. The measurement electrode 44 corresponds to the measurement electrode. The first and second diffusion rate-limiting sections 11 and 13 correspond to the diffusion rate-limiting sections.

In the sensor element 101 included in the gas sensor 100 according to the present embodiment described in detail above, the sensor element 101 is configured such that the average height t of the first and second diffusion rate-limiting sections 11 and 13, obtained using Equation (1), is 0.0035 mm or greater. This configuration allows suppression of an excessive increase in the diffusion resistance of the predetermined portion of the gas path from the outside up to the inner pump electrode 22, thereby preventing an excessive increase in the static pressure dependence of the pump current Ip0 of the main pump cell 21. As a result, it is possible to provide a sensor element 101 capable of suppressing an excessive increase in the static pressure dependence of the pump current Ip0.

Additionally, in the sensor element 101, by configuring it such that the average height t of the first and second diffusion rate-limiting sections 11 and 13, as obtained using Equation (1), is 0.0090 mm or greater, the excessive increase in the diffusion resistance of the predetermined portion can be further suppressed, and the excessive increase in the static pressure dependence of the pump current Ip0 can also be further suppressed.

Furthermore, in the sensor element 101, by configuring it such that the average height t of the first and second diffusion rate-limiting sections 11 and 13, as obtained using Equation (1), is 0.0250 mm or less, the excessive increase in the deterioration rate of the inner pump electrode 22 can be suppressed.

It should be noted that the present invention is not limited to the present embodiment described above in any way, and it goes without saying that the present invention can be implemented in various modes as long as they fall within the technical scope of the present invention.

For example, in the embodiment described above, the inner pump electrode 22 and the auxiliary pump electrode 51 are formed as porous cermet electrodes composed of Pt containing 1% Au and ZrO₂, and the outer pump electrode 23, the reference electrode 42, and the measurement electrode 44 are all formed as porous cermet electrodes composed of Pt and ZrO₂. However, one or more of the electrodes 22, 23, 42, 44, and 51 may not be cermet. In addition, one or more of the electrodes 22, 23, 42, 44, and 51 may not be porous body.

In the embodiment described above, the oxygen concentration adjustment chamber included the first internal cavity 20 and the second internal cavity 40, but the present invention is not limited thereto. For example, the oxygen concentration adjustment chamber may further include another internal cavity, or one of the first internal cavity 20 and the second internal cavity 40 may be omitted. Similarly, in the embodiment described above, the adjustment pump cell included the main pump cell 21 and the auxiliary pump cell 50, but the present invention is not limited thereto. For example, the adjustment pump cell may further include another pump cell, or one of the main pump cell 21 and the auxiliary pump cell 50 may be omitted. For example, if the oxygen concentration of the measurement gas can be sufficiently lowered by the main pump cell 21 alone, the auxiliary pump cell 50 may be omitted. When the auxiliary pump cell 50 is omitted, the control unit 96 only needs to perform the main pump control processing as the adjustment pump control processing. In addition, in the main pump control processing, the setting of the target value V0* based on the pump current Ip1 described above may be omitted. Specifically, a predetermined target value V0* is stored in the storage unit 98 in advance, and the control unit 96 controls the voltage Vp0 of the variable power source 24 by feedback control such that the voltage V0 reaches the target value V0*, thereby controlling the main pump cell 21. In this case, for example, the oxygen partial pressure Pod can be obtained by acquiring the voltage V0 and the oxygen partial pressure Podr of the reference gas (air) when the main pump control processing is being performed, and applying these to a predetermined correspondence relationship between the voltage V0, the oxygen partial pressure Podr, and the oxygen partial pressure Pod.

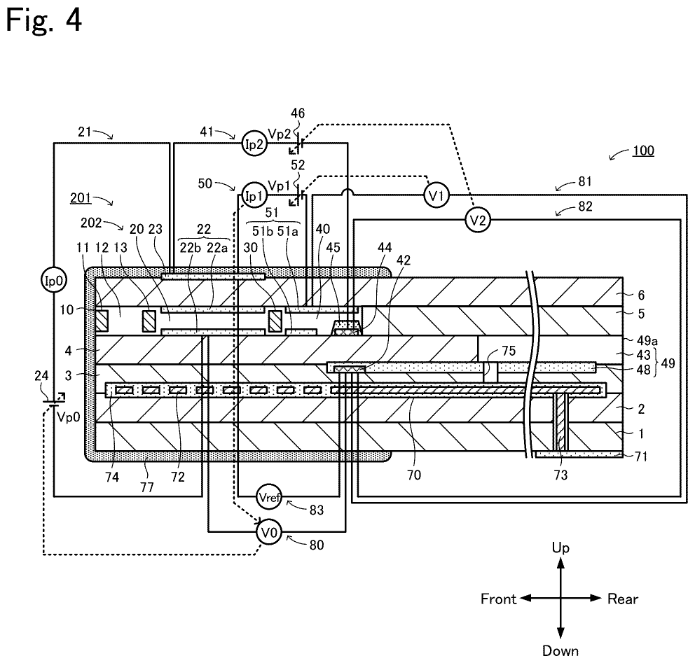

In the embodiment described above, the element body 102 of the sensor element 101 of the gas sensor 100 includes the first internal cavity 20, the second internal cavity 40, and the third internal cavity 61. However, this is not limited to such a configuration. For example, as shown in FIG. 4, an element body 202 of a modified sensor element 201 may not include the third internal cavity 61. In the element body 202 of the modified sensor element 201 shown in FIG. 4, between the lower surface of the second solid electrolyte layer 6 and the upper surface of the first solid electrolyte layer 4, the following components are adjacently formed and in communication with each other, in the order listed: a gas inlet 10; a first diffusion rate-limiting section 11; a buffer space 12; a second diffusion rate-limiting section 13; a first internal cavity 20; a third diffusion rate-limiting section 30; and a second internal cavity 40. In addition, the measurement electrode 44 is disposed on the upper surface of the first solid electrolyte layer 4 within the second internal cavity 40. The measurement electrode 44 is covered with a fourth diffusion rate-limiting section 45, which is a film made of a porous ceramic material such as alumina (Al₂O₃). The fourth diffusion rate-limiting section 45 serves to limit the amount of NOx flowing to the measurement electrode 44, similarly to the fourth diffusion rate-limiting section 60 of the embodiment described above. Furthermore, the fourth diffusion rate-limiting section 45 also functions as a protective film for the measurement electrode 44. The ceiling electrode portion 51a of the auxiliary pump electrode 51 extends directly above the measurement electrode 44. Even with such a configuration of the sensor element 201, the NOx concentration can be detected based on, for example, the pump current Ip2, similarly to the embodiment described above. In this case, the region around the measurement electrode 44 functions as the measurement chamber.

In the embodiment described above, the outer pump electrode 23 serves multiple functions: as an electrode (also referred to as the outer main pump electrode) paired with the inner pump electrode 22 in the main pump cell 21; as an electrode (also referred to as the outer auxiliary pump electrode) paired with the auxiliary pump electrode 51 in the auxiliary pump cell 50; and as an electrode (also referred to as the outer measurement electrode) paired with the measurement electrode 44 in the measurement pump cell 41. However, this is not limited to such a configuration. For example, at least one of the outer main pump electrode, the outer auxiliary pump electrode, and the outer measurement electrode may instead be provided separately from the outer pump electrode 23 and arranged on the outer side of the element body 102, in contact with the measurement gas.

In the embodiment described above, the element body 102 of the sensor element 101 is configured to have its front end portion covered with the porous protective layer 77. However, the front end portion may alternatively be exposed without the porous protective layer 77. The inventors have confirmed through experiments and analysis that when the front end portion of the element body 102 is not covered with the porous protective layer 77, the static pressure dependence of the pump current Ip0, provided that the average height t of the first and second diffusion rate-limiting sections 11 and 13 are the same, is lower compared to when the front end portion is covered with the porous protective layer 77. Therefore, even when the front end portion of the element body 102 is not covered with the porous protective layer 77, the average height t of the first and second diffusion rate-limiting sections 11 and 13 needs to be 0.0035 mm or greater, just as in the case where the front end portion is covered with the porous protective layer 77. In the case where the front end portion of the element body 102 is not covered with the porous protective layer 77, compared to when it is covered, when the other parameters of the main pump cell 21 (such as the path lengths L1 and L2, and the widths H1 and H2 of the first and second diffusion rate-limiting sections 11 and 13) except the limiting current Ip of the main pump cell 21 are the same as in Equation (1), the diffusion resistance from the outside up to the first internal cavity 20 decreases, which increases the limiting current Ip of the main pump cell 21, and the average height t of the first and second diffusion rate-limiting sections 11 and 13 obtained using Equation (1) increases.

In the embodiment described above, the element body 102 of the sensor element 101 includes the first diffusion rate-limiting section 11 and the second diffusion rate-limiting section 13. However, this is not limited to such a configuration. Either the first diffusion rate-limiting section 11 or the second diffusion rate-limiting section 13 may be omitted, leaving only the other section. Alternatively, another diffusion rate-limiting section may be included. For example, in a case where the second diffusion rate-limiting section 13 is omitted and only the first diffusion rate-limiting section 11 is included, the height t of the first diffusion rate-limiting section 11 is obtained using Equation (3). In a case where the first through n-th diffusion rate-limiting sections are included, the average height t of these sections is obtained using Equation (4) based on the respective path lengths Li (i: 1 to n) and widths Hi of the first through n-th diffusion rate-limiting sections. Equation (4) is derived from the right-hand side of Equation (2), where “L/H” is replaced with “ΣLi/Hi”.

$$t = L 1 / H 1 \times Ip \times 1 / ( 4 \times F \times D / ( R \times T ) ) \times 1 / ( Poe - Pod ) \times 10 \qquad ( 3 )$$

$$t = Σ Li / Hi \times Ip \times 1 / ( 4 \times F \times D / ( R \times T ) ) \times 1 / ( Poe - Pod ) \times 10 \qquad ( 4 )$$

In the embodiment described above, the first diffusion rate-limiting section 11 of the element body 102 of the sensor element 101 includes an upper slit 11a and a lower slit 11b, but it may include only one of these slits. Similarly, the second diffusion rate-limiting section 13 includes an upper slit 13a and a lower slit 13b, but it may include only one of these slits.

In the embodiment described above, the sensor element 101 is configured to detect the NOx concentration in the measurement gas. However, this is not limited to such a configuration, and it may be used to detect the concentration of the specific gas in the measurement gas. For example, the specific gas concentration may be an oxide concentration other than NOx. In a case where the specific gas is an oxide, oxygen is generated through the reduction of the specific gas itself in the third internal cavity 61, as in the above-described embodiment. Accordingly, the measurement pump cell 41 can detect the concentration of the specific gas by acquiring a detection value (e.g. the pump current Ip2) corresponding to the amount of oxygen thus generated. Alternatively, the specific gas may be a non-oxide such as ammonia. In this case, the specific gas is first converted into an oxide (e.g., ammonia into NO), and oxygen is thereby generated through the reduction of the converted gas in the third internal cavity 61. Accordingly, the measurement pump cell 41 can detect the concentration of the specific gas by acquiring a detection value (e.g. the pump current Ip2) corresponding to the oxygen generated in this process. For example, the inner pump electrode 22 located in the first internal cavity 20 may function as a catalyst for converting ammonia into NO within the first internal cavity 20.

In the embodiments described above, the element body 102 of the sensor element 101 is formed as the laminated body including multiple solid electrolyte layers (layers 1 to 6). However, this is not limited to such a configuration. The element body 102 of the sensor element 101 only needs to include at least one oxygen ion-conductive solid electrolyte layer. For example, in FIG. 1, layers 1 to 5, except for the second solid electrolyte layer 6, may be formed of materials other than solid electrolyte (such as alumina). In such a case, each electrode included in the sensor element 101 may be disposed on the second solid electrolyte layer 6. For instance, the measurement electrode 44 shown in FIG. 1 may be disposed on a lower surface of the second solid electrolyte layer 6. Furthermore, the reference gas introduction space 43, which is formed in the first solid electrolyte layer 4, may instead be formed in the spacer layer 5. Likewise, the reference gas introduction layer 48, located between the first solid electrolyte layer 4 and the third substrate layer 3, may instead be provided between the second solid electrolyte layer 6 and the spacer layer 5. In addition, the reference electrode 42 may be provided on the lower surface of the second solid electrolyte layer 6 at a position downstream of the third internal cavity 61.

In the embodiments described above, the control unit 96 performs feedback control by setting a target value V0* for the voltage V0 based on the pump current Ip1, such that the pump current Ip1 reaches a target value Ip1*. The control unit then adjusts the pump voltage Vp0 such that the voltage V0 reaches the target value V0*. However, other control methods may also be employed. For example, the control unit 96 may perform feedback control of the pump voltage Vp0 based on the pump current Ip1, such that the pump current Ip1 reaches the target value Ip1*. In other words, the control unit 96 may bypass acquiring the voltage V0 from the oxygen partial pressure detection sensor cell 80 used for main pump control and setting the target value V0*, and instead directly control the pump voltage Vp0 (and thus the pump current Ip0) based on the pump current Ip1.

In the embodiment described above, the configuration of the sensor element 101 included in the gas sensor 100 has been described. Next, an evaluation method for the sensor element 101 will be described. First, a sensor element 101 to be evaluated and a corresponding temperature measurement sample are manufactured using the manufacturing method described above, and the path lengths L1 and L2 and the widths H1 and H2 of the first and second diffusion rate-limiting sections 11 and 13 at this time are obtained. The path lengths L1 and L2 and the widths H1 and H2 may be, for example, design values, or they may be measured by manufacturing multiple identical sensor elements 101 and cutting a portion of them. Subsequently, for the sensor element 101 to be evaluated and the corresponding temperature measurement sample, the limiting current Ip of the main pump cell 21, the temperature T of the inner pump electrode 22, the oxygen partial pressure Poe in the measurement gas, and the oxygen partial pressure Pod in the first internal cavity 20 are obtained using the measurement method described above. Then, a computer inputs various data related to the sensor element 101 to be evaluated, calculates the average height t of the first and second diffusion rate-limiting sections 11 and 13 using Equation (1) described above, and evaluates using the calculated average height t of the first and second diffusion rate-limiting sections 11 and 13. As described above, Equation (1) is derived from Equation (2). Specifically, the evaluation determines whether the average height t of the first and second diffusion rate-limiting sections 11 and 13 is 0.0035 mm or greater. This configuration allows evaluation of whether it is possible to suppress the excessive increase in the static pressure dependence of the pump current Ip0. As a result, it is possible to provide a sensor element 101 capable of suppressing the excessive increase of the static pressure dependence of the pump current Ip0. Instead of evaluating whether the average height t of the first and second diffusion rate-limiting sections 11 and 13 is 0.0035 mm or greater, it is also possible to evaluate whether the average height t of the first and second diffusion rate-limiting sections 11 and 13 is 0.0090 mm or greater. In addition to evaluating whether the average height t of the first and second diffusion rate-limiting sections 11 and 13 is 0.0035 mm or greater or whether it is 0.0090 mm or greater, it is also possible to evaluate whether the average height t of the first and second diffusion rate-limiting sections 11 and 13 is 0.0250 mm or less. In this way, it is also possible to evaluate whether the excessive increase in the deterioration rate of the main pump cell 21, specifically of the inner pump electrode 22, can be suppressed. As a result, it is also possible to provide a sensor element 101 capable of suppressing an excessive increase in the deterioration rate of the inner pump electrode 22.

It should be noted that for the sensor element 101 to be evaluated, the computer performs the processing of calculating the average height t of the first and second diffusion rate-limiting sections 11 and 13, and the processing of evaluating using the calculated average height t of the first and second diffusion rate-limiting sections 11 and 13. However, at least some of these processes may be performed by a person.

The evaluation method for the sensor element 101 has been described. However, the processing of the evaluation method for the sensor element 101, specifically, the processing of calculating the average height t of the first and second diffusion rate-limiting sections 11 and 13 for the sensor element 101 to be evaluated, and the processing of evaluating using the calculated average height t of the first and second diffusion rate-limiting sections 11 and 13, may also be implemented as a program executed by one or more computers. The program may be recorded on a computer-readable recording medium (e.g. a hard disk, SSD, ROM, FD, CD, DVD, etc.), may be distributed from one computer to another via a transmission medium (such as a communication network like the Internet or LAN), or may be transferred in other forms.

### EXAMPLE

Specifically fabricated examples of the sensor element will be described as examples. It should be noted that the present invention is not limited to the following examples.

### Experimental Examples 1 to 140

Using the manufacturing method described above, a sensor element 101 as shown in FIG. 1 was produced and designated as Experimental Example 1. In addition, the temperature measurement sample 1 corresponding to Experimental Example 1 was also produced. In producing the sensor element 101, a ceramic green sheet was formed by mixing zirconia particles with 4 mol % of stabilizing agent yttria, an organic binder, and an organic solvent, followed by tape molding. Using the same manufacturing method, Experimental Examples 2 to 140 and temperature measurement samples 2 to 140 corresponding to Experimental Examples 2 to 140 were also produced. In Experimental Examples 1 to 140 and temperature measurement samples 1 to 140, the path lengths L1 and L2, the widths H1 and H2, and the heights t11, t12, t21, and t22 of the upper slits 11a and 13a and the lower slits 11b and 13b of the first and second diffusion rate-limiting sections 11 and 13 were varied.

Subsequently, for Experimental Example 1 and the corresponding temperature measurement sample 1, the limiting current Ip of the main pump cell 21, the temperature T of the inner pump electrode 22, the oxygen partial pressure Poe in the measurement gas, and the oxygen partial pressure Pod in the first internal cavity 20 were obtained using the measurement method described above. Then, for Experimental Example 1, the average height t of the first and second diffusion rate-limiting sections 11 and 13 was calculated using Equation (1) described above. Similarly, for Experimental Examples 2 to 140, the average height t of the first and second diffusion rate-limiting sections 11 and 13 was calculated in the same manner. The average height t of the first and second diffusion rate-limiting sections 11 and 13 in Experimental Examples 1 to 140 ranged from 0.0031 to 0.0202.

### [Evaluation Test]

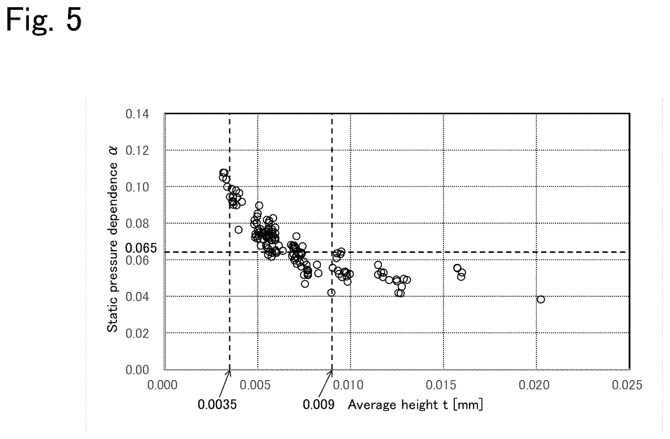

The gas sensor 100 including the sensor element 101 of Experimental Example 1 was installed to a pipe such that the tip-side portion of the sensor element 101 protruded into the inside of the pipe. Then, electric power was supplied to the heater 72 to heat the sensor element 101 to a temperature of 800° C. In this state, a model gas was prepared, in which the base gas was nitrogen, the oxygen concentration was 18%, the specific gas (NOx) concentration was 0 ppm, and the pressure was 101.3 kPa. This model gas was then introduced into the pipe as a first measurement gas. Then, the adjustment pump control processing (the auxiliary pump control processing using the target value V1*, and the main pump control processing) described above was performed to acquire the pump current Ip0 of the main pump cell 21 as the first pump current Ipa. Next, a model gas was prepared, in which the base gas was nitrogen, the oxygen concentration was 18%, the specific gas (NOx) concentration was 0 ppm, and the pressure was 150.3 kPa. This model gas was then introduced into the pipe as a second measurement gas. Then, the adjustment pump control processing was performed to acquire the pump current Ip0 of the main pump cell 21 as the second pump current Ipb. After obtaining the first and second pump currents Ipa and Ipb in this manner, a static pressure dependence index α indicating the static pressure dependence of the pump current Ip0 was calculated using Equation (5) based on the obtained pump currents Ipa and Ipb. Similarly, the static pressure dependence index α was calculated for Experimental Examples 2 to 140 as well. Then, a graph showing the relationship between the average height t of the first and second diffusion rate-limiting sections 11 and 13 and the static pressure dependence index α for Experimental Examples 1 to 140 is shown in FIG. 5.

$$α = ( 1 - Ipa / Ipb ) / ( 1 - 101.3 kPa / 150 kPa ) \qquad ( 5 )$$

In the evaluation test, when the static pressure dependence index α is 0.10 or less, it is determined that the static pressure dependence of the pump current Ip0 is favorable (not excessively high), and when the static pressure dependence index α is greater than 0.10, it is determined that the static pressure dependence of the pump current Ip0 is not favorable (excessively high). As shown in FIG. 5, among Experimental Examples 1 to 140, in those in which the average height t of the first and second diffusion rate-limiting sections 11 and 13 is 0.0035 mm or greater, the static pressure dependence index α is 0.10 or less. Further, in those in which the average height t of the first and second diffusion rate-limiting sections 11 and 13 is 0.0090 mm or greater, the static pressure dependence index α is 0.065 or less. Therefore, in sensor elements 101 in which the average height t of the first and second diffusion rate-limiting sections 11 and 13 is 0.0035 mm or greater, it can be determined that the static pressure dependence of the pump current Ip0 is favorable, and among these, in sensor elements 101 in which the average height t of the first and second diffusion rate-limiting sections 11 and 13 is 0.0090 mm or greater, it can be determined that the static pressure dependence of the pump current Ip0 is more favorable.

What is claimed is:

1. A sensor element configured to detect a concentration of a specific gas in a measurement gas, the sensor element comprising:
an element body having an oxygen-ion-conductive solid electrolyte layer and a measurement gas flow path provided therein, the measurement gas flow path configured to introduce and circulate the measurement gas;
at least one adjustment pump cell having an inner electrode disposed in an oxygen concentration adjustment chamber within the measurement gas flow path, the adjustment pump cell being configured to adjust an oxygen concentration in the oxygen concentration adjustment chamber;
a measurement electrode disposed in a measurement chamber located downstream of the oxygen concentration adjustment chamber within the measurement gas flow path; and
a diffusion rate-limiting section provided in the measurement gas flow path, the diffusion rate-limiting section being configured to guide the measurement gas from the outside into the oxygen concentration adjustment chamber while imparting diffusion resistance thereto;
wherein a height t [mm] of the diffusion rate-limiting section, obtained using Equation (A) based on the following parameters: a path length L [cm] of the diffusion rate-limiting section; a width H [cm] of the diffusion rate-limiting section; a limiting current Ip [A] of the adjustment pump cell; the Faraday constant F [A·sec/mol]; the diffusion coefficient D [cm²/sec] of oxygen; the gas constant R [cm³·atm/mol K]; a temperature T [K] of the inner electrode; an oxygen partial pressure Poe [atm] in the measurement gas; and an oxygen partial pressure Pod [atm] in the oxygen concentration adjustment chamber, is 0.0035 or greater,
$$t = L / H \times Ip \times 1 / ( 4 \times F \times D / ( R \times T ) ) \times 1 / ( Poe - Pod ) \times 10. \qquad ( A )$$

2. The sensor element according to claim 1,
wherein the height t is 0.0090 or greater.

3. The sensor element according to claim 1,
wherein the height t is 0.0250 or less.

4. The sensor element according to claim 1,
wherein the diffusion rate-limiting section includes first to n-th (n≥2) diffusion rate-limiting sections,
the L/H is obtained as a sum of Li/Hi, using path lengths Li (i: 1 to n) [cm] and widths Hi [cm] of the respective first to n-th diffusion rate-limiting sections, and
the height t is an average of the heights ti of the respective first to n-th diffusion rate-limiting sections.

5. The sensor element according to claim 1,
wherein the adjustment pump cells, each having the oxygen concentration adjustment chamber and the inner electrode, are provided in series along the measurement gas flow path,
the diffusion rate-limiting section is provided upstream of the most upstream oxygen concentration adjustment chamber within the measurement gas flow path, and
the limiting current Ip is the limiting current of the adjustment pump cell that adjusts the oxygen concentration in the most upstream oxygen concentration adjustment chamber within the measurement gas flow path.

6. A gas sensor comprising:
the sensor element according to claim 1.

7. An evaluation method for a sensor element configured to detect a concentration of a specific gas in a measurement gas, the sensor element comprising:
an element body having an oxygen-ion-conductive solid electrolyte layer and a measurement gas flow path provided therein, the measurement gas flow path configured to introduce and circulate the measurement gas;
at least one adjustment pump cell having an inner electrode disposed in an oxygen concentration adjustment chamber within the measurement gas flow path, the adjustment pump cell being configured to adjust an oxygen concentration in the oxygen concentration adjustment chamber;
a measurement electrode disposed in a measurement chamber located downstream of the oxygen concentration adjustment chamber within the measurement gas flow path; and
a diffusion rate-limiting section provided in the measurement gas flow path, the diffusion rate-limiting section being configured to guide the measurement gas from the outside into the oxygen concentration adjustment chamber while imparting diffusion resistance thereto;
wherein the evaluation method for the sensor element under evaluation executes:
(a) a step of calculating a height t [mm] of the diffusion rate-limiting section using Equation (B) based on a path length L [cm] of the diffusion rate-limiting section, a width H [cm] of the diffusion rate-limiting section, a limiting current Ip [A] of the adjustment pump cell, the Faraday constant F [A·sec/mol], the diffusion coefficient D [cm²/sec] of oxygen, the gas constant R [cm³ atm/mol·K], a temperature T [K] of the inner electrode, an oxygen partial pressure Poe [atm] in the measurement gas, and an oxygen partial pressure Pod [atm] in the oxygen concentration adjustment chamber; and
(b) a step of evaluating using the height t,
$$t = L / H \times Ip \times 1 / ( 4 \times F \times D / ( R \times T ) ) \times 1 / ( Poe - Pod ) \times 10. \qquad ( B )$$

8. A program for causing one or more computers to execute each step of the evaluation method for the sensor element according to claim 6.
